(19) United States
(12) Patent Application Publication
(10) Pub. No.: US 2014/0039333 A1
(43) Pub. Date: Feb. 6, 2014

(54) SYSTEMS AND METHODS FOR DETECTING MECHANICAL DYSSYNCHRONY AND STROKE VOLUME FOR USE WITH AN IMPLANTABLE MEDICAL DEVICE EMPLOYING A MULTI-POLE LEFT VENTRICULAR LEAD

(71) Applicant: Xiaoyi Min, Thousand Oaks, CA (US)

(72) Inventor: Xiaoyi Min, Thousand Oaks, CA (US)

(73) Assignee: PACESETTER, INC., Sylmar, CA (US)

(21) Appl. No.: 13/563,356

(22) Filed: Jul. 31, 2012

(51) Int. Cl. A61N 1/365; A61B 5/0452

(52) U.S. Cl. 600/510; 607/18

(57) ABSTRACT

Techniques are provided for use with an implantable medical device for evaluating mechanical cardiac dyssynchrony based impedance (Z) measured along different vectors between an electrode in the right ventricle (RV) and various electrodes of a multi-pole left ventricle (LV) lead.

### TECHNICAL FIELD

The invention generally relates to implantable cardiac rhythm management devices such as pacemakers, implantable cardioverter-defibrillators (ICDs) and cardiac resynchronization therapy (CRT) devices and, in particular, to techniques for detecting mechanical cardiac dyssynchrony and stroke volume using implantable devices equipped with multi-pole leads.

### BACKGROUND

Heart failure is a debilitating disease in which abnormal function of the heart leads to inadequate blood flow to fulfill the needs of the tissues and organs of the body. Typically, the heart loses propulsive power because the cardiac muscle loses capacity to stretch and contract. Often, the ventricles do not adequately fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result. Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds cardiac muscle causing the ventricles to grow in volume in an attempt to pump more blood with each heartbeat, i.e. to increase the stroke volume. This places a still higher demand on the heart's oxygen supply. If the oxygen supply falls short of the growing demand, as it often does, further injury to the heart may result, typically in the form of myocardial ischemia or myocardial infarction. The additional muscle mass may also stiffen the heart walls to hamper rather than assist in providing cardiac output. A particularly severe form of heart failure is congestive heart failure (CHF) wherein the weak pumping of the heart leads to build-up of fluids in the lungs and other organs and tissues. Heart failure is often associated with mechanical cardiac dyssynchrony, i.e. the inability of the chambers of the heart to contract synchronously, particularly the left and right ventricles, and further associated with a drop in stroke volume.

In view of the potential severity of heart failure, it is desirable to detect its onset within a patient and track its progression so that appropriate therapy can be provided. Many patients suffering heart failure already have pacemakers, ICDs or CRT devices implanted therein or are candidates for such devices. Accordingly, it is desirable to provide such devices with the capability to automatically detect and track heart failure. Information regarding mechanical dyssynchrony and stroke volume within the heart of the patient can be used in the detection and tracking of heart failure, as well as in the setting of pacing delays, particularly atrioventricular (AV), interventricular (VV) and inter-electrode pacing delays for use with CRT. Accordingly, it would be desirable to provide techniques for detecting and tracking mechanical cardiac dyssynchrony and stroke volume and it is to these ends that various aspects of the invention are directed.

### SUMMARY

In an exemplary embodiment, a method is provided for use with an implantable medical device having a lead system including a right ventricular (RV) lead and a multi-pole left ventricular (LV) lead implanted via the coronary sinus (CS). Signals representative of impedance (Z) are measured along different RV-LV vectors between an electrode in the RV (such as the RV coil) and the various electrodes of the multi-pole LV lead (e.g. LV tip, LV mid 1, LV mid 2 and LV prox 4.) The signals are measured throughout one or more cardiac cycles to track cardiogenic variations in impedance associated with the contraction and expansion of heart muscle. Reference points are identified within the time-varying signals for each of the different RV-LV vectors, such as respective maximum (Zmax) and minimum (Zmin) points, or max dZ/dt points. The relative timings of corresponding reference points are determined for the different RV-LV vectors, such as the relative times (t) of the various Zmax points and the relative times (t′) of the various Zmin points. Values representative of mechanical dyssynchrony are then determined from a comparison of the relative timings of corresponding reference points within the various time-varying signals, such as by determining standard deviations of the timing values derived from all four RV-LV vectors. Then, one or more functions of the implantable device are controlled based on the values representative of mechanical dyssynchrony, such as by recording diagnostic data indicative of the amount of mechanical dyssynchrony, generating warning signals indicative of heart failure, selecting electrodes or setting pacing delays for use with CRT in an effort to reduce or eliminate the dyssynchrony. Stroke volume may additionally or alternatively be assessed for use in detecting heart failure or setting pacing delays.

In an illustrative embodiment, the implantable system includes a CRT device equipped with a quadripolar LV lead and an RV lead with tip, ring and coil electrodes. Impedance is measured along each of four RV-LV vectors: an LV distal tip-RV coil vector; an LV mid1 ring-RV coil vector; an LV mid2 ring-RV coil vector; and an LV prox 4-RV coil vector. (In other examples, the RV ring is instead be used rather than the RV coil.) Hence, four time-varying impedance signals are measured (Z₁, Z₂, Z₃, Z₄) corresponding to the four RV-LV vectors. Within each vector, two reference points are identified within each cardiac cycle, Zmax and Zmin, and the times of those points are determined relative to one another or relative to the QRS complex of a corresponding intracardiac electrogram (IEGM.) The times of the Zmax points of the four RV-LV vectors are denoted t₁, t₂, t₃, t₄; the times of the Zmin points are denoted t′₁, t′₂, t′₃, t′₄. The device then assesses LV systolic dyssynchrony (S_Dys) based on the standard deviation of the set of Zmax time values (t₁, t₂, t₃, t₄.) In general, the greater the standard deviation in the distribution of the Zmax values, the greater the degree of systolic dyssynchrony. The device also assesses LV diastolic dyssynchrony (D_Dys) based on the standard deviation of the set of Zmin time values (t′₁, t′₂, t′₃, t′₄.) In general, the greater the standard deviation in the Zmin values, the greater the degree of diastolic dyssynchrony. Additionally or alternatively, cross-correlation among the four RV-LV vector impedance signals may also be determined as a measure of LV mechanical dyssynchrony, with good correlation indicating LV synchrony and with poor correlation indicating LV dyssynchrony. Heart failure may then be detected and tracked based on increasing LV mechanical dyssynchrony. Suitable warning signals may be generated to alert the patient or caregiver and diagnostic data may be stored for clinician review. VV pacing delay values and LV inter-electrode pacing delays for use with CRT may be set, at least in part, based on the amount of LV mechanical dyssynchrony. In this manner, RV-LV impedance vectors are used to assess LV mechanical dyssynchrony to aid in clinical diagnosis and the setting of certain pacing delays.

In the illustrative embodiment, the device also uses certain RV impedance measurements, in combination RV-LV impedance measurements, to assess interventricular dyssynchrony. To this end, impedance is also measured along an RV vector between a pair of RV electrodes, such as RV tip-RV ring or RV tip-RV coil. Within the RV vector, Zmax and Zmin reference points are identified and the timing of those points relative to corresponding points within one of the RV-LV vectors (such as the Z₁ vector) is determined. The time of the Zmax point within the RV vector is herein denoted trv; whereas the time of the Zmin point is denoted trv′. The device then assesses interventricular dyssynchrony (VV_Dys) based on the difference between try and the corresponding t value of the selected RV-LV vector (such as t₁ of Z₁) and/or based on the difference between trv′ and the corresponding t′ value of the selected RV-LV vector (such as t′₁ of Z₁.) In general, the larger the difference between the timing of the reference point in the RV impedance vector and the corresponding reference point in the RV-LV impedance vector, the greater the interventricular dyssynchrony. AV and VV pacing delay values may be set, at least in part, based on the amount of the mechanical dyssynchrony so as to reduce or minimize the dyssynchrony. Also in the illustrative embodiment, the device uses the Zmax and Zmin values to assess stroke volume and to adjust the AV and VV values to increase or maximize stroke volume.

In the various examples described herein, impedance measurements are used but it should be understood that related electrical parameters might be detected and/or exploited instead, such as admittance, conductance or immittance. Those skilled in the art can convert between these related parameters as needed and where appropriate. Accordingly, herein, “values representative of impedance” is deemed to include related electrical parameters such as admittance, conductance and immittance. Also, in addition to RV coil (or ring) to LV impedance vectors, techniques described herein are applicable to other vectors such as current injected from RV coil to LV tip with voltage sensed from RV tip or RV ring to RV coil. Note also that values representative of mechanical dyssynchrony obtained via impedance can be exploited along with values representative of electrical dyssynchrony obtained via analysis of an IEGM to determine preferred or optimal pacing delay values, to select preferred or optimal LV electrodes or to achieve other goals or purposes. Still further, an assessment of mechanical dyssynchrony (M_DS) derived from impedance can be combined with an assessment of stroke volume (SVZ) derived from impedance to set preferred or optimal pacing delay values that achieve both acceptable SVZ and acceptable M_DS.

System and method implementations of the various exemplary embodiments are presented herein.

### BRIEF DESCRIPTION OF THE DRAWINGS

Features and advantages of the described implementations can be more readily understood by reference to the following description taken in conjunction with the accompanying drawings.

FIG. 1 illustrates pertinent components of an implantable medical system having a pacemaker, ICD or CRT device equipped to detect mechanical dyssynchrony and stroke volume based on impedance signals detected using a multi-pole LV lead;

FIG. 2 provides an overview of the technique for detecting mechanical dyssynchrony based on impedance that may be performed by the system of FIG. 1;

FIG. 3 illustrates changes in cardiogenic impedance measured along different RV-LV vectors, which may be exploited by the method of FIG. 2;

FIG. 4 illustrates an exemplary technique for use with the general method of FIG. 2 for detecting and responding to LV mechanical dyssynchrony based on a set of RV-LV impedance vectors;

FIG. 5 illustrates variations in mechanical dyssynchrony for different values of AV delay, demonstrating that changes in AV delay can significantly affect mechanical dyssynchrony, which can be exploited by the method of FIG. 4;

FIG. 6 illustrates another exemplary technique for use with the general method of FIG. 2 for detecting and responding to both LV mechanical dyssynchrony and interventricular dyssynchrony based on RV-LV impedance vectors and RV vectors;

FIG. 7 is a simplified, partly cutaway view, illustrating the device of FIG. 1 along with at set of leads implanted in or on the heart of the patient;

FIG. 8 is a functional block diagram of the device of FIG. 7, illustrating basic circuit elements that provide cardioversion, defibrillation and/or pacing stimulation in the heart and particularly illustrating on-board components for performing the various mechanical dyssynchrony evaluation techniques; and

FIG. 9 is a functional block diagram illustrating components of the external device programmer of FIG. 1 and particularly illustrating programmer-based optimization components for controlling the various mechanical dyssynchrony evaluation techniques.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable Medical System

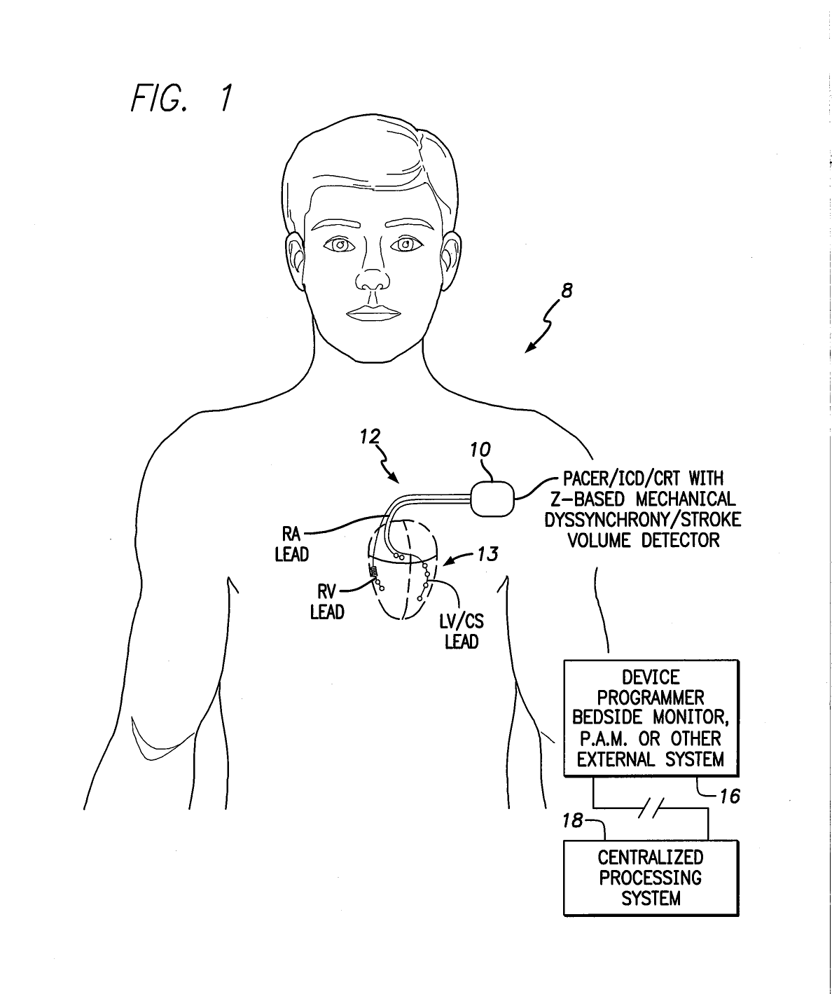

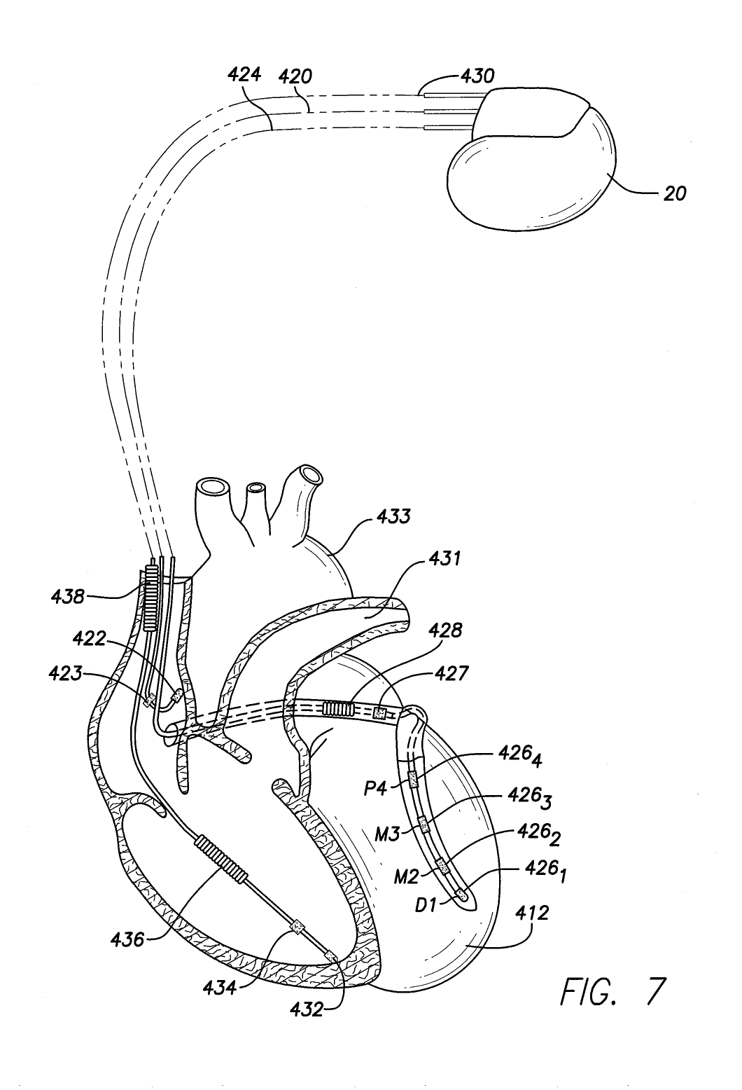

FIG. 1 illustrates an implantable cardiac rhythm management system 8 capable of detecting mechanical cardiac dyssynchrony and stroke volume based on impedance measured using a multi-pole LV lead and an RV lead. The implantable system 8 includes a pacer/ICD/CRT device 10 equipped with one or more leads 12 implanted on or within the heart of the patient, including a multi-pole LV lead implanted via the coronary sinus (CS). To illustrate the multi-pole configuration of the LV lead, a set of electrodes 13 is shown distributed along the LV lead. In the examples described herein, a quadripolar (or “quad-pole”) lead is employed (such as the Quartet™ lead provided by St Jude Medical). Other suitable LV leads may instead be employed, including leads with more or fewer electrodes. Exemplary RV and RA leads are also shown that include tip/ring pairs. The RV lead includes an RV coil, which can be used as an RV reference electrode in combination with the various LV electrodes to provide a set of RV-LV impedance vectors. Other electrodes of various sizes and shapes may be additionally or alternatively provided, such as coil electrodes mounted in or on the superior vena cava (SVC), the LV or the left atrium (LA.) See FIG. 7 for a more complete illustration of the configuration of an exemplary lead system. Although identified as a pacer/ICD/CRT in FIG. 1, it should be understood that device 10 can be any suitably-equipped implantable medical device such as a standalone pacemaker, ICD or CRT device, including CRT-D and CRT-P devices. In the following, for brevity, device 10 will be referred to simply as a CRT device.

Based in the assessment of mechanical dyssynchrony and stroke volume, the CRT device can optimize pacing delays and/or detect and track heart failure or related conditions using techniques described below. Depending upon the particular conditions detected, the device will issue warning signals, if appropriate. For example, if a significant progression of heart failure is indicated based on changes in mechanical dyssynchrony, warning signals may be generated to warn the patient, either using an internal warning device (which can be part of the CRT device) or using an external bedside monitor/handheld warning device 16 or other external system. The internal warning device may be a vibrating device or a “tickle” voltage device that, in either case, provides perceptible stimulation to the patient to alert the patient so that the patient may consult a physician. In one example, once the warning is felt, the patient positions an external warning device above his or her chest. The handheld device, which might be a personal advisory module (PAM), receives short-range telemetry signals from the implanted device and provides audible or visual verification of the warning signal. The handheld warning device thereby provides confirmation of the warning to the patient, who might otherwise be uncertain as to the reason for the internally generated warning signal. For further information regarding this warning/notification technique, see U.S. Pat. No. 7,272,436 to Gill et al.

If a bedside monitor is provided, the bedside monitor provides audible or visual alarm signals to alert the patient or caregivers, as well as providing textual or graphic displays. In addition, any diagnostic information pertaining to the deteriorating cardiac condition of the patient is transferred to the bedside monitor or is stored within the CRT device for subsequent transmission to an external programmer for review by a clinician or other medical professional. The clinician may then prescribe therapies to address the condition. The clinician may also adjust the operation of the CRT device to activate, deactivate or otherwise control any therapies that are automatically applied, including titration of medications if an implantable drug infusion pump is provided. The bedside monitor may be directly networked with an internet network site or a centralized processing system 18 for immediately notifying the clinician of any urgent medical condition. The centralized system may include such systems as Merlin.Net of St. Jude Medical, which may be used in conjunction with bedside monitors or similar devices such as the HouseCall™ remote monitoring system or the Merlin@home systems, also of St. Jude Medical.

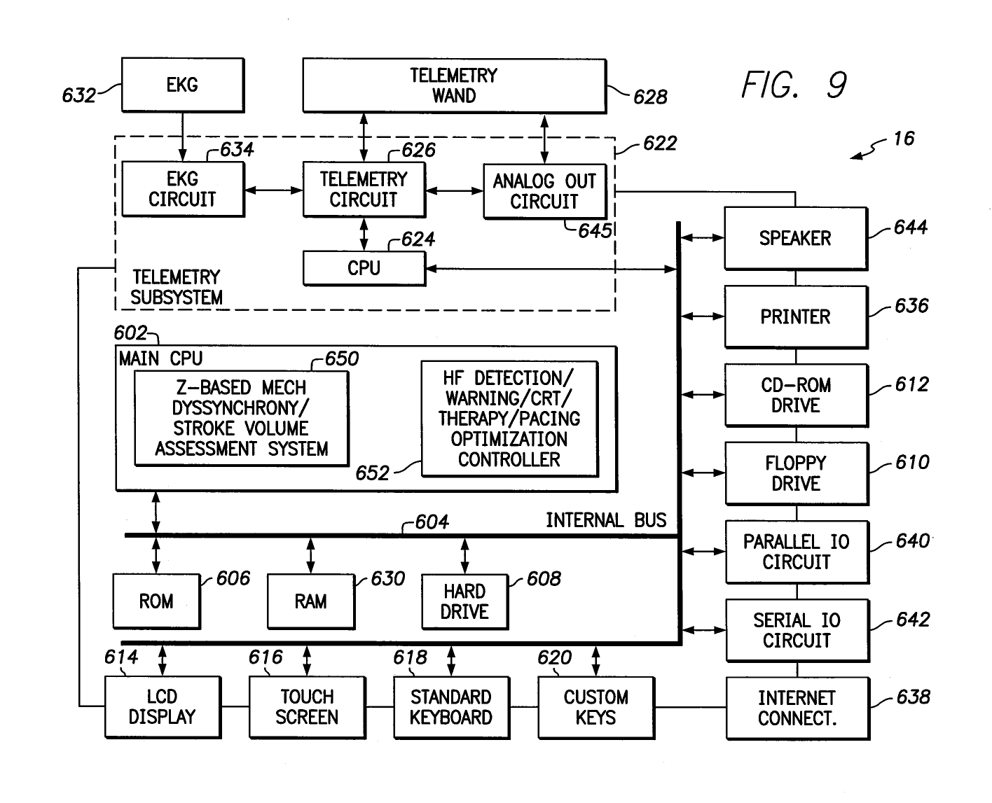

In some implementations, the CRT device itself assesses mechanical dyssynchrony based on impedance measurements made using its leads. In other implementations, the device transmits impedance measurements to the external system 16, which performs the assessment. In the following examples, it is assumed that the CRT device performs the assessment using on-board components. An example where the external programmer performs the assessment is described below with reference to FIG. 9.

Hence, FIG. 1 provides an overview of an implantable medical system for assessing mechanical dyssynchrony and stroke volume, optimizing pacing delays, detecting and tracking heart failure, and delivering appropriate warning/notification signals and therapy, etc. Embodiments may be implemented that do not necessarily perform all of these functions. For example, embodiments may be implemented that assess mechanical dyssynchrony but do not automatically adjust therapy. In addition, note that the particular locations of the implanted components shown in FIG. 1 are merely illustrative and may not necessarily correspond to actual implant locations.

### Overview of Mechanical Dyssynchrony Assessment Systems/Techniques

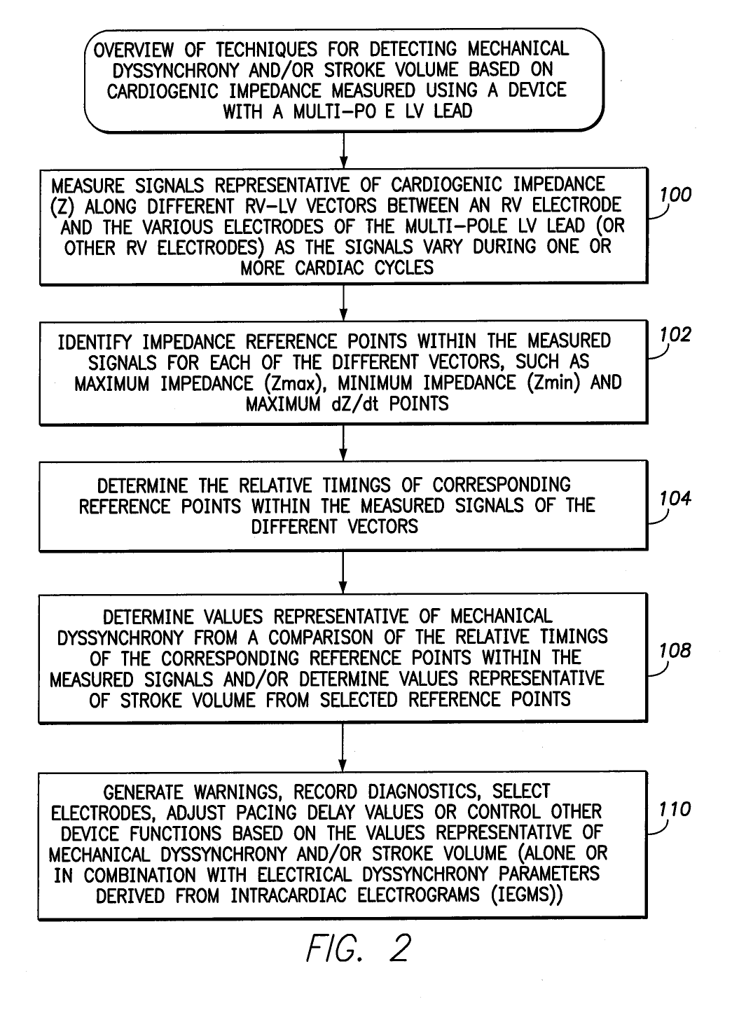

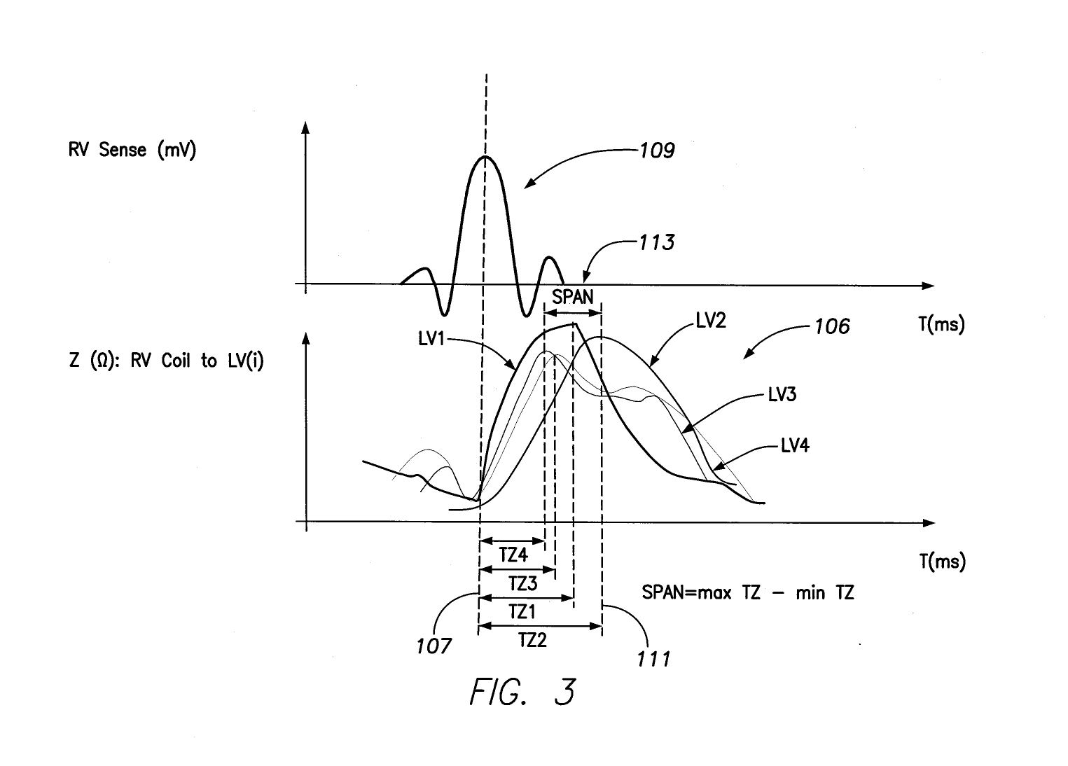

FIGS. 2 and 3 broadly summarize a general method of assessing mechanical dyssynchrony and stroke volume based on impedance that may be exploited by the components of the system of FIG. 1. Beginning at step 100, the CRT device measures signals representative of cardiogenic impedance (such as impedance (Z), admittance, conductance or immittance) along different RV-LV vectors between an RV electrode (such as the RV coil) and the various electrodes of the multi-pole LV lead as the signals vary during one or more cardiac cycles. That is, a set of time-varying impedance signals Z(t) are measured, with one Z(t) signal measured for each of the different RV-LV vectors. For a quadripolar LV lead, four RV-LV vectors are thereby measured, yielding four corresponding Z(t) signals, denoted Z₁, Z₂, Z₃ and Z₄ (or c1, c2, c3 and c4.) Note that various RV-RV impedance vectors may also be exploited such as by injecting current RV coil to LV tip and then sensing voltage RV tip or RV ring to RV coil. At step 102, the device identifies reference points (or fiducial points) within the measured Z signals for each of the different RV-LV vectors (and/or any RV-RV impedance vectors that are used), such as maximum impedance (Zmax), minimum impedance (Zmin) and max dZ/dt points. Hence, in one example, each separate impedance vector (e.g. Z₁) yields a pair of Zmax and Zmin values (e.g. Z₁ max and Z₁ min) for a given cardiac cycle, as well as a max dZ/dt value. At step 104, the device determines the relative timings of corresponding reference points within the measured signals of the different RV-LV vectors (and/or any RV-RV impedance vectors that are used). For example, the device determines the time (t1) of the Zmax value of the first vector, the time (t2) of the Zmax value of the second vector, and so on. Likewise, the device determines the time (t1′) of the Zmin value of the first vector, the time (t2′) of the Zmin value of the second vector, and so on.

FIG. 3 provides a graph 106 showing four exemplary cardiogenic Z(t) signal traces (LV1, LV2, LV3 and LV4) derived from RV coil to LV and the timing of the corresponding Zmax points within each of the traces. In the figure, time 107 corresponds to the peak of an RV sense signal 109. Alternatively, other reference points or landmarks using far field IEGM or ECG instead of RV IEGM can be used. Time intervals between the peak of RV QRS and subsequent Zmax points (such as time 111) for each of the four vectors are shown and denoted TZ1, TZ2, TZ3 and TZ4, respectively. As can be seen, the various time intervals differ significantly in this test subject, indicating a significant degree of mechanical dyssynchrony. The largest difference or “span” between the TZ values (i.e. the difference between the maximum TZ value and the minimum TZ value) is denoted in the figure as “SPAN” 113 and is a measure of the degree of mechanical dyssynchrony. The greater the value for the span, the greater the dyssynchrony. Note also that, although FIG. 3 only illustrates timing to max Z, similar methods apply to timing to max dZ/dt or min Z. For example, the time from the peak of RV QRS to max dZ/dt can be used to assess dyssynchrony. Also, from the same signals of Z, stroke volume (SVZ) can be obtained where SVZ=max Z−min Z.

Returning to FIG. 2, at step 108, the CRT device determines values representative of mechanical dyssynchrony from a comparison of the relative timings (t, t′) of the corresponding reference points (Zmax, Zmin) within the measured signals and/or determines values representative of stroke volume from selected reference values (particularly the difference between max Z and min Z.) Techniques for detecting or estimating both systolic and diastolic mechanical dyssynchrony, as well as stroke volume, are discussed below. At step 110, the device generates warnings, records diagnostics, selects electrodes, adjusts pacing delay values and/or controls other device functions based on the values representative of mechanical dyssynchrony and/or stroke volume. This may be performed alone or in combination with electrical dyssynchrony parameters derived from IEGMs. That is, the values representative of mechanical dyssynchrony obtained via impedance can be exploited along with values representative of electrical dyssynchrony obtained via analysis of an IEGM to determine preferred or optimal pacing delay values, to select preferred or optimal LV electrodes, or to achieve other goals or purposes. Various patents describing IEGM-based optimization techniques are listed below. Still further, an assessment of mechanical dyssynchrony (M_DS) derived from impedance can be combined with an assessment of stroke volume (SVZ) derived from impedance to set preferred or optimal pacing delay values that achieve both acceptable SVZ and acceptable M_DS.

Thus, FIGS. 2 and 3 broadly summarize techniques for assessing or evaluating mechanical dyssynchrony and/or stroke volume based on impedance measured using a multi-pole LV lead. These techniques will be described in greater detail in the following sections.

### Exemplary Mechanical Dyssynchrony Assessment Techniques

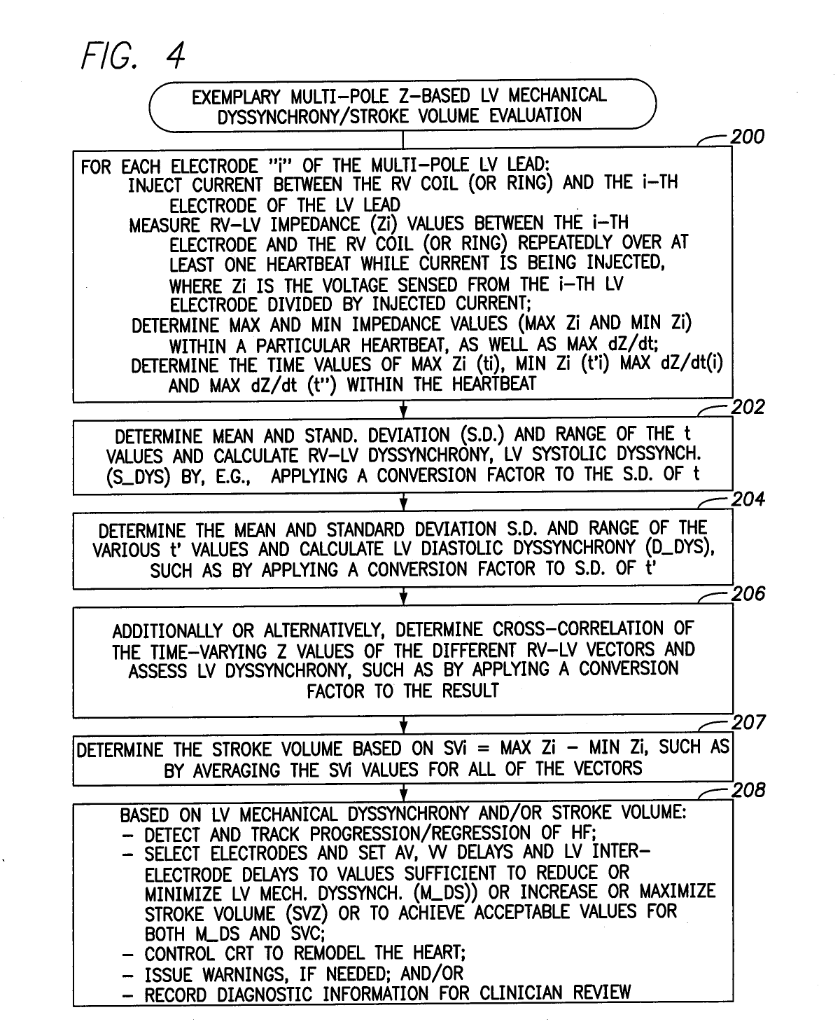

FIG. 4 illustrates a first illustrative technique for detecting mechanical dyssynchrony using a lead configuration exploiting a multi-pole LV lead. In this example, LV dyssynchrony is assessed. In a subsequent example, both LV dyssynchrony and interventricular dyssynchrony are assessed. At step 200 of FIG. 4, for each electrode “i” of the multi-pole LV lead, the CRT device: injects current between the RV coil (or RV ring) and the i-th electrode of the LV lead; measures RV-LV impedance (Zi) values between the i-th electrode and the RV coil (or RV ring) continuously over at least one heartbeat, where Zi is the voltage sensed from the RV coil (or RV ring) to the i-th LV electrode divided by the injected current; determines maximum and minimum impedance values (max Zi and min Zi) within each heartbeat as well as max dZ/dt(i); and then determines the time value of max Zi (denoted ti) within the heartbeat and determines the time value of min Zi (denoted t′i) within the heartbeat and the time value for max dZ/dt (denoted t″i). The time values may be measured relative to the peak of the QRS complex of a corresponding IEGM or relative to some other appropriate reference (fiducial point) within the cardiac cycle. For a quadripole LV example were the RV coil is used, the device therefore measures impedance along four vectors (RV coil-LV distal tip (or D1), RV coil-LV mid 2 (or M2), RV coil-LV mid 3 (or M3) and RV coil-LV prox 4 (or P4)) throughout each heartbeat while current is being applied and then determines four sets of max impedance values (max Z_{RV-D1}, max Z_{RV-M2}, max Z_{RV-M3}, max Z_{RV-P4}) and four sets of min impedance values (min Z_{RV-D1}, min Z_{RV-M2}, min Z_{RV-M3}, min Z_{RV-P4}) within each heartbeat. As can be appreciated, data may be collected over multiple heartbeats (i.e. multiple cardiac cycles) and then averaged together.

At step 202, the device determines the mean and standard deviation and range of the various t values and calculates RV-LV dyssynchrony, LV systolic dyssynchrony (S_DYS) or other useful parameters therefrom. (More specifically, mean is used to assess RV-LV dyssynchrony; whereas the standard deviation and the range are used for intra-LV dyssynchrony.) In general, the greater the standard deviation or range in the distribution of the timing of the Zmax values, the greater the degree of systolic dyssynchrony. Note that, for many purposes, it is not necessary to calculate a calibrated (i.e. absolute) value of the amount of systolic dyssynchrony. Rather, it is often sufficient to track changes in S_DYS over time so as to detect increasing (or decreasing) dyssynchrony. Accordingly, the value of the standard deviation of the t values can be used as the numerical value for S_DYS for tracking purposes. For applications where a calibrated value for systolic dyssynchrony might be needed, the device can apply a predetermined scaling factor to convert the standard deviation value to a calibrated value of S_DYS, where the conversion factor is determined in advance based on a comparison of measured standard deviation values (derived from the t values) with known values for systolic dyssynchrony obtained within the patient using suitable evaluation techniques such as Tissue Doppler echocardiography. Different conversion factors might be determined for use with different patient postures, heart rates, etc. Likewise, at step 204, the device determines the mean and standard deviation and range of the various t′ values and then calculates LV diastolic dyssynchrony (D_DYS) therefrom. The greater the standard deviation or range in the distribution of the timing of the Zmax or equivalence values, the greater the degree of diastolic dyssynchrony. For applications where a calibrated value for D_DYS might be needed, the device can apply a predetermined conversion factor to convert the standard deviation value to a calibrated value of D_DYS.

Additionally or alternatively, at step 206, the device determines the numerical cross-correlation of the various time-varying Z values of the different RV-LV vectors and assesses LV dyssynchrony therefrom. In particular, step 206 may be used as an alternative to steps 202 and 204. Otherwise conventional cross-correlation calculation techniques can be applied to cross-correlate the time-varying Z₁(t) signal for a given cardiac cycle with the time-varying Z₂(t) signal for the same cardiac cycle and with the other time-varying Zᵢ(t) signals for each of the RV-LV vectors. As with the S_Dys and D_Dys values, for any applications where a calibrated value for LV dyssynchrony might be needed, the device can apply a predetermined conversion factor to convert the cross-correlation value to a calibrated measure of LV dyssynchrony. At step 207, the device determines the stroke volume (SV) based on SVi=max Zi−min Zi, such as by summing the SVi values for all of the vectors. Note that when stroke value is estimated, the relative timing of the points of max Z and min Z is not used to calculate SV. Rather, the magnitude of the values for max Z and min Z are used (i.e. the amount above or below a reference value of Z=0.)

Within steps 202-206, when using max dZ/dt, the relative timing of the max dZ/dt values for each vector can be compared to the timing of the max dZ/dt values for the other vectors to assess an amount of mechanical dyssynchrony. As noted above, the time delay from the peak of QRS to max dZ/dt can be used or the time delay to min dZ/dt or to max dZ/dt can be used. The various measures of dyssynchrony (derived from max Z or min Z or max dZ/dt) can be averaged or otherwise combined to generate a combined dyssynchrony value or “metric” for use in assessing dyssynchrony, tracking heart failure and controlling device functions.

At step 208, the device then exploits the various LV dyssynchrony values such as S_Dys and D_Dys and/or the stroke volume values to: detect and track progression/regression of heart failure; select electrodes and optimize AV, VV delays and LV inter-electrode pacing delays to minimize or otherwise reduce LV mechanical dyssynchrony (M_DS) and to maximize or other increase stroke volume (SVZ) or to achieve acceptable values for both M_DS and SVC; control CRT to remodel the heart; issue warnings, if needed, perhaps in response to a significant progression of heart failure; and/or record diagnostic information for clinician review. Detection of heart failure may be indicated based on the LV mechanical dyssynchrony exceeding a corresponding threshold value or stroke volume falling below a corresponding threshold value. Subsequent progression of heart failure may be indicated based on a significant increase in LV mechanical dyssynchrony over time or a significant decrease in stroke volume over time.

Insofar as the optimization of pacing delays is concerned, AV, VV and/or LV inter-electrode pacing delay values may be adjusted while monitoring LV mechanical dyssynchrony and stroke volume to determine delay values sufficient to minimize (or at least reduce) LV mechanical dyssynchrony or heart failure or sufficient to maximize (or at least increase) stroke volume. If both mechanical dyssynchrony (M_DS) and Z-based stroke volume (SVC) are used for determination of optimal delays, the following procedure may be used. Define an acceptable range of M_DS (i.e. Min M_DS+x %*the range of M_DS) and that of SVZ (=max SVZ−y %*the range of SVZ. Then, the delays that have M_DS and SVZ in the acceptable range can be selected as the “optimal” delays.

Optimization may be performed in conjunction with other optimization techniques. See, for example, the following patents and patent applications that set forth various systems and methods (including IEGM-based methods) for determining and/or adjusting pacing delays: U.S. Pat. No. 7,590,446 of Min et al.; U.S. Published Patent Application 2009/0299423; U.S. patent application Ser. No. 11/952,743, filed Dec. 7, 2007, entitled “Systems and Methods for Determining Optimal Atrio-Ventricular Pacing Delays using either Paced or Sensed Atrial Beats” (Attorney Docket No. A07P1179); U.S. Published Patent Application 2010/0145405, entitled Systems and Methods for Controlling Ventricular Pacing in Patients with Long Intra-Atrial Conduction Delays” (Attorney Docket No. A08P1067); U.S. Published Patent Application 2011/0022110 of Min et al., entitled “Systems and Methods for Optimizing Ventricular Pacing Delays for use with Multi-Pole Leads” (Attorney Docket No. A09P1034); U.S. Published Patent Application 2011/0022112 of Min et al., entitled “Systems and Methods for Determining Ventricular Pacing Sites for use with Multi-Pole Leads” (Attorney Docket No. A09P1034US01); U.S. Pat. No. 8,145,311 of Min et al., entitled “Systems and Methods for Determining Optimal Electrode Pairs for use in Biventricular Pacing using Multi-Pole Ventricular Leads” (Attorney Docket No. A09P1053); U.S. Published Patent Application 2012/0136406, of Min, entitled “Systems and Methods for Determining Optimal Atrioventricular Pacing Delays based on Cardiomechanical Delays” (Attorney Docket No. A10P1051); and U.S. patent application Ser. No. 12/976,322, filed Dec. 22, 2010, 2010, of Min et al., entitled “Systems and Methods for Optimizing AV/VV Pacing Delays using Combined IEGM/Impedance-based Techniques for use with Implantable Medical Devices” (Attorney Docket No. A10P1055). See, also, U.S. Pat. No. 7,248,925, to Bruhns et al., entitled “System and Method for Determining Optimal Atrioventricular Delay based on Intrinsic Conduction Delays.” See, also, U.S. patent application Ser. No. 13/023,408 of Min, filed Feb. 8, 2011, entitled “Systems and Methods for Tracking Stroke Volume Using Hybrid Impedance Configurations Employing a Multi-Pole Implantable Cardiac Lead.” At least some of the techniques are implemented within the QuickOpt™ systems of St. Jude Medical.

It should be understood that the “optimal” delays obtained using the techniques described herein are not necessarily absolutely optimal in a given quantifiable or mathematical sense. What constitutes “optimal” depends on the criteria used for judging the resulting performance, which can be subjective in the minds of some clinicians. The pacing delays determined by the techniques described herein represent, at least, “preferred” delays. Clinicians may choose to adjust or alter the selection of the delays for particular patients, at their discretion.

As noted, CRT techniques may be employed in an effort to remodel the heart to improve mechanical synchrony. Briefly, CRT seeks to normalize asynchronous cardiac electrical activation and resultant asynchronous contractions associated with heart failure by delivering synchronized pacing stimulus to both ventricles. The stimulus is synchronized so as to improve overall cardiac function. This may have the additional beneficial effect of reducing the susceptibility to life-threatening tachyarrhythmias. CRT is discussed, for example, in U.S. Pat. No. 8,019,409 to Rosenberg et al., entitled “Cardiac Resynchronization Therapy Optimization using Electromechanical Delay from Realtime Electrode Motion Tracking” and in U.S. Published Patent Application 2008/0306567 of Park et al., entitled “System and Method for Improving CRT Response and Identifying Potential Non-Responders to CRT Therapy.” See, also, U.S. Patent Application 2010/0114232 of Min, entitled “Initiation Tests and Guidelines for Implementing Cardiac Therapy.”

Insofar as the diagnostic information to be recorded for clinician review is concerned, the device can record the estimated LV mechanical dyssynchrony values (e.g. S_DYS and D_DYS), as well as any of the intermediate impedance values determined by the device (such as the various maximum or minimum impedance values and their relative timing.) This information may be recorded along with device operational data (such as the current pacing configuration, pacing rate, etc.) and patient physiological/anatomical data (such as current posture, heart rate, blood pressure, etc.), assuming such information is available.

As already explained, rather than detecting impedance, other related electrical signals or parameters can instead be exploited, such as admittance, conductance, immittance or their equivalents, where appropriate. See, also, the near-field impedance techniques set forth in: U.S. Published Patent Application 2012/0035493, of Gutfinger et al., entitled “Near Field-Based Systems and Methods for Assessing Impedance and Admittance for use with an Implantable Medical Device” (Atty. Docket No. A10P1031) and related applications. Also, as mentioned, besides RV coil (or ring) to LV impedance vectors, techniques described herein are applicable to other vectors such as current injected from RV coil to LV tip with voltage sensed from RV tip or RV ring to RV coil.

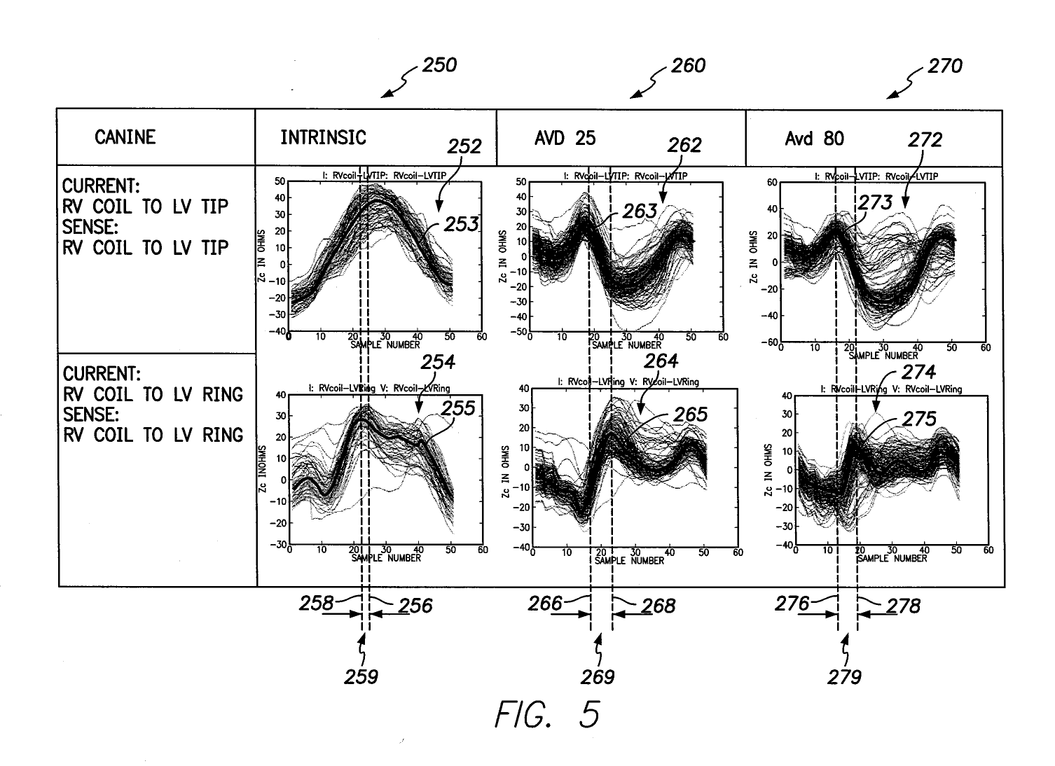

FIG. 5 illustrates how changes in AV delay values can affect mechanical dyssynchrony within an otherwise healthy canine test subject. More specifically, a first pair of figures 250 shows impedance tracings without any AV delay applied to the test subject where an upper graph 252 shows Z measured with current applied RV coil to LV tip and sensed RV coil to LV tip (with curve 253 representing the average over beats) and where a lower graph 254 shows Z measured RV coil to LV ring and sensed RV coil to LV ring (with curve 255 representing the average.) Within each of the two graphs, individual traces correspond to different heart beats. The average timing for max Z within graph 252 is shown by line 256; whereas the average timing for max Z within graph 254 is shown by line 258. The span 259 between the two (i.e. the difference between the two max Z values for the two vectors) is also shown. For the healthy subject, the span is relatively small, indicating good synchrony. A second pair of figures 260 shows similar impedance tracings with AV delay set to 25 ms where again an upper graph 262 shows Z measured with current applied RV coil to LV tip and sensed RV coil to LV tip (with curve 263 representing the average) and where a lower graph 264 shows Z measured RV coil to LV ring and sensed RV coil to LV ring (with curve 265 representing the average.) The average timing for max Z within graph 262 is shown by line 266; whereas the average timing for max Z within graph 254 is shown by line 258. The span 269 between the two is also shown and indicates a greater amount of mechanical dyssynchrony. A third pair of graphs 270 shows similar impedance tracings with AV delay set to 80 ms where again an upper graph 272 shows Z measured with current applied RV coil to LV tip and sensed RV coil to LV tip (with curve 273 representing the average) and where a lower graph 274 shows Z measured RV coil to LV ring and sensed RV coil to LV ring (with curve 275 representing the average.) The average timing for max Z within graph 272 is shown by line 276; whereas the average timing for max Z within graph 274 is shown by line 278. The span 279 between the two is also shown and indicates a still greater amount of mechanical dyssynchrony. Although these traces are for a healthy test subject where an increase in AV delay causes mechanical dyssynchrony, the opposite effect should occur in a subject with heart failure. That is, for a patient with mechanical dyssynchrony, adjustments in AV delay should cause changes in the amount of mechanical dyssynchrony and, in particular, proper selection of the AV delay should reduce or substantially eliminate the mechanical dyssynchrony.

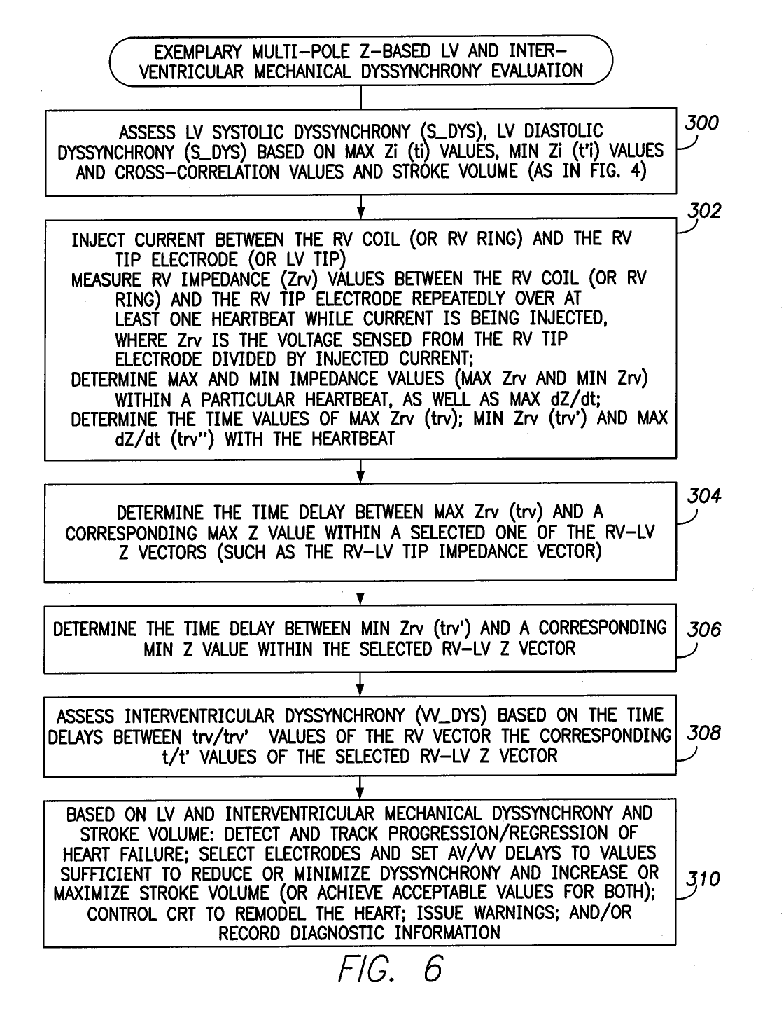

FIG. 6 illustrates a second illustrative technique for detecting mechanical dyssynchrony using a lead system exploiting a multi-pole LV lead. In this example, both LV dyssynchrony and interventricular (VV) dyssynchrony are assessed. Some of the steps are the same or similar to those already described and hence will not be described in detail again. At step 300, the device assesses LV systolic dyssynchrony (S_DYS) and LV diastolic dyssynchrony (S_DYS) based on max Zi (ti) values, min Zi (t′i) values, etc., and cross-correlation values and detects stroke volume (as already described in connection with FIG. 4.) At step 302, the device then begins to assess interventricular dyssynchrony by: injecting current between the RV coil (or RV ring) and the RV tip electrode of the RV lead (or LV tip); measuring RV impedance (denoted Zrv) values between the RV coil (or ring) and the RV tip repeatedly over at least one heartbeat where Zrv is the voltage sensed from the RV tip electrode divided by the injected current; determining maximum and minimum impedance values (max Zrv and min Zrv) within each heartbeat, as well as the time value for max dZ/dt; and then determining the time value of max Zrv (denoted trv) with the heartbeat and the time value of min Zrv (denoted trv′) with the heartbeat, as well as the time value for max dZ/dt (denoted trv″i).

At step 304, the device determines the time delay between max Zrv (trv) and a corresponding max Z value within a selected one of the RV-LV Z vectors (such as the RV-LV D1 impedance vector, Z₁.) At step 306, the device determines the time delay between min Zrv (trv′) and a corresponding min Z value within the selected RV-LV Z vector. At step 308, the device assesses interventricular dyssynchrony (VV_DYS) based on the time delays between trv/trv′ values of the RV vector the corresponding t/t′ values of the selected RV-LV Z vector. For example, the device can determine the time delay between max Zrv (i.e. trv) and max Z₁ (i.e. t₁) for use as a value representative of interventricular dyssynchrony. Additionally or alternatively, the device can determine the time delay between min Zrv (i.e. trv′) and min Z₁ (i.e. t′₁) for use as the value representative of interventricular dyssynchrony. If both values are determined, both values may be exploited. Additionally or alternatively, time delays between the max and min reference points in the RV impedance signal and corresponding max and min points within other RV-LV vectors (such as Z₂ or Z₃) can be used to provide a further measure of interventricular dyssynchrony. In general, the greater the timing difference between the reference points in the RV impedance signal and the corresponding reference points in the RV-LV impedance signals, the greater the degree of interventricular mechanical dyssynchrony.

As with the LV dyssynchrony values discussed above, for many purposes it is not necessary to calculate a calibrated (i.e. absolute) measure of interventricular dyssynchrony. Rather, it is often sufficient to track changes in interventricular dyssynchrony over time so as to detect increasing (or decreasing) dyssynchrony. Accordingly, the time delay values detected at steps 304 and 306 can be used as numerical values for interventricular dyssynchrony for tracking purposes. For applications where a calibrated value for VV_DYS might be needed, the device can apply a predetermined conversion factor to convert the time delays to a calibrated value of VV_DYS.

At step 310, the device exploits the various LV and interventricular dyssynchrony values such as S_DYS, D_DYS and VV_DYS, as well as estimated stroke volume to: detect and track progression/regression of heart failure; select electrodes and optimize AV/PV/VV delays and LV inter-electrode pacing delays to minimize or otherwise reduce LV mechanical dyssynchrony and interventricular dyssynchrony and/or to maximize or otherwise increase stroke volume (or achieve acceptable values for both as discussed above); control CRT to remodel the heart; issue warnings, if needed, perhaps in response to a significant progression of heart failure; and/or record diagnostic information for clinician review. Detection and progression of heart failure may be indicated based on changes in stroke volume, LV mechanical dyssynchrony and/or interventricular dyssynchrony over time.

Depending upon the particular implementation, some or all of the steps of the various figures are performed by the implantable device itself. Additionally or alternatively, at least some of the steps can be performed by an external programmer or other external system based on impedance or other data measured within the patient and then transmitted to the external device. Also, although primarily described with respect to examples having a CRT device equipped with a quadripole lead, other implantable medical devices and lead systems may instead be equipped to exploit the techniques described. For the sake of completeness, an exemplary CRT device will now be described, which includes components for performing the functions and steps already described.

Exemplary CRT Device with Quadripole Lead

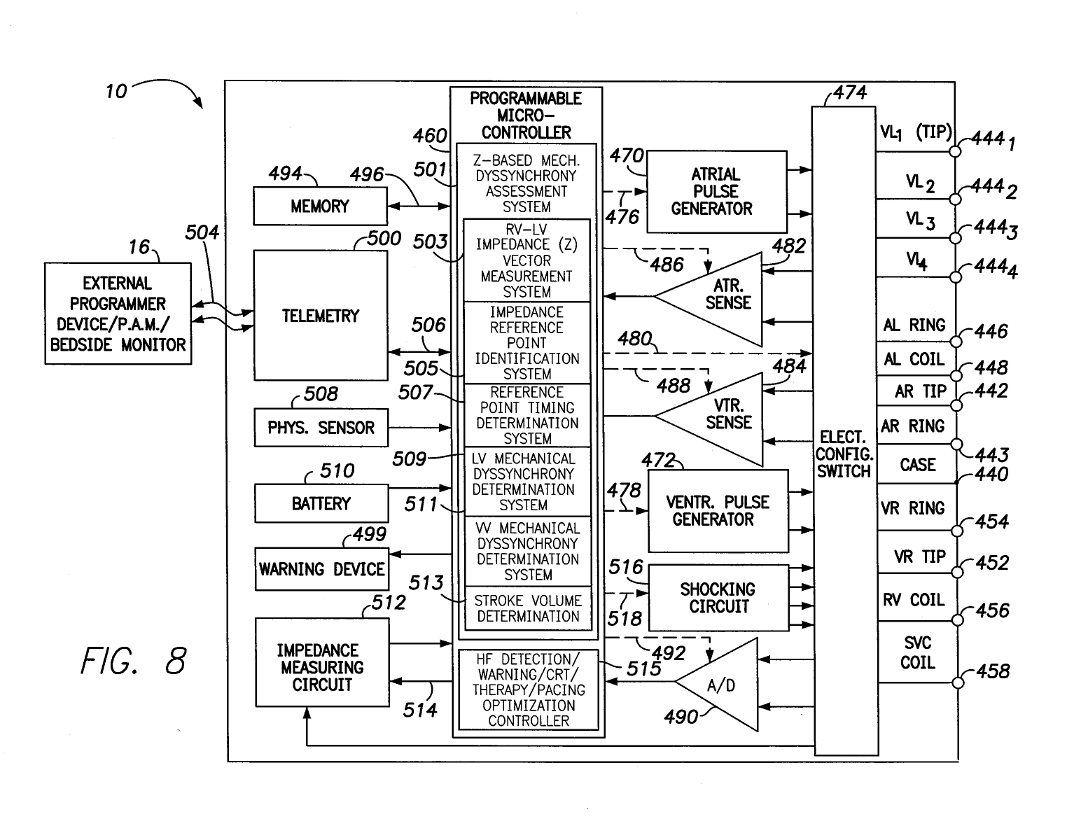

With reference to FIGS. 7 and 8, a description of an exemplary CRT device will now be provided. FIG. 7 provides a simplified block diagram of the device, which is a dual-chamber stimulation device capable of treating both fast and slow arrhythmias with stimulation therapy, including cardioversion, defibrillation, and pacing stimulation, and also capable of assessing mechanical dyssynchrony, as discussed above, and controlling functions in response thereto, such as CRT. To provide other atrial chamber pacing stimulation and sensing, device 10 is shown in electrical communication with a heart 412 by way of a right atrial lead 420 having an atrial tip electrode 422 and an atrial ring electrode 423 implanted in the atrial appendage. Device 10 is also in electrical communication with the heart by way of a right ventricular lead 430 having, in this embodiment, a ventricular tip electrode 432, a right ventricular ring electrode 434, a right ventricular (RV) coil electrode 436, and a superior vena cava (SVC) coil electrode 438. Typically, the right ventricular lead 430 is transvenously inserted into the heart so as to place the RV coil electrode 436 in the right ventricular apex, and the SVC coil electrode 438 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, device 10 is coupled to a multi-pole LV lead 424 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 424 is designed to receive atrial and ventricular cardiac signals and to deliver left ventricular pacing therapy using a set of four left ventricular electrodes 426₁, 426₂, 426₃, and 426₄ (thereby providing a quadripole lead), left atrial pacing therapy using at least a left atrial ring electrode 427, and shocking therapy using at least a left atrial coil electrode 428 implanted on or near the left atrium. In other examples, more or fewer LV electrodes are provided. Although only three leads are shown in FIG. 7, it should be understood that additional leads (with one or more pacing, sensing and/or shocking electrodes) might be used and/or additional electrodes might be provided on the leads already shown, such as additional electrodes on the RV lead.

A simplified block diagram of internal components of device 10 is shown in FIG. 7. While a particular device is shown, this is for illustration purposes only and one of skill in the art could readily duplicate, eliminate or disable the appropriate circuitry in any desired combination to provide a device capable of treating the appropriate chamber(s) with cardioversion, defibrillation and pacing stimulation. The housing 440 for device 10, shown schematically in FIG. 8, is often referred to as the “can,” “case” or “case electrode” and may be programmably selected to act as the return electrode for all “unipolar” modes. The housing 440 may further be used as a return electrode alone or in combination with one or more of the coil electrodes, 428, 436 and 438, for shocking purposes. The housing 440 further includes a connector (not shown) having a plurality of terminals, 442, 443, 444₁-444₄, 446, 448, 452, 454, 456 and 458 (shown schematically and, for convenience, the names of the electrodes to which they are connected are shown next to the terminals). As such, to achieve right atrial sensing and pacing, the connector includes at least a right atrial tip terminal (A_{R} TIP) 442 adapted for connection to the atrial tip electrode 422 and a right atrial ring (A_{R} RING) electrode 443 adapted for connection to right atrial ring electrode 423. To achieve left chamber sensing, pacing and shocking, the connector includes a left ventricular tip terminal (VL₁ TIP) 444₁ and additional LV electrode terminals 444₂-444₄ for the other LV electrodes of the quadra-pole LV lead.

The connector also includes a left atrial ring terminal (A_{L} RING) 446 and a left atrial shocking terminal (A_{L} COIL) 448, which are adapted for connection to the left atrial ring electrode 427 and the left atrial coil electrode 428, respectively. To support right chamber sensing, pacing and shocking, the connector further includes a right ventricular tip terminal (V_{R} TIP) 452, a right ventricular ring terminal (V_{R} RING) 454, a right ventricular shocking terminal (V_{R} COIL) 456, and an SVC shocking terminal (SVC COIL) 458, which are adapted for connection to the right ventricular tip electrode 432, right ventricular ring electrode 434, the V_{R} coil electrode 436, and the SVC coil electrode 438, respectively.

At the core of device 10 is a programmable microcontroller 460, which controls the various modes of stimulation therapy. As is well known in the art, the microcontroller 460 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 460 includes the ability to process or monitor input signals (data) as controlled by a program code stored in a designated block of memory. The details of the design and operation of the microcontroller 460 are not critical to the invention. Rather, any suitable microcontroller 460 may be used that carries out the functions described herein. The use of microprocessor-based control circuits for performing timing and data analysis functions are well known in the art.

As shown in FIG. 8, an atrial pulse generator 470 and a ventricular pulse generator 472 generate pacing stimulation pulses for delivery by the right atrial lead 420, the right ventricular lead 430, and/or the LV lead 424 via an electrode configuration switch 474. It is understood that in order to provide stimulation therapy in each of the four chambers of the heart, the atrial and ventricular pulse generators, 470 and 472, may include dedicated, independent pulse generators, multiplexed pulse generators or shared pulse generators. The pulse generators, 470 and 472, are controlled by the microcontroller 460 via appropriate control signals, 476 and 478, respectively, to trigger or inhibit the stimulation pulses.

The microcontroller 460 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 474 includes a plurality of switches for connecting the desired electrodes to the appropriate I/O circuits, thereby providing complete electrode programmability. Accordingly, the switch 474, in response to a control signal 480 from the microcontroller 460, determines the polarity of the stimulation pulses (e.g., unipolar, bipolar, combipolar, etc.) by selectively closing the appropriate combination of switches (not shown) as is known in the art. The switch also switches among the various LV electrodes.

Atrial sensing circuits 482 and ventricular sensing circuits 484 may also be selectively coupled to the right atrial lead 420, LV lead 424, and the right ventricular lead 430, through the switch 474 for detecting the presence of cardiac activity in each of the four chambers of the heart. Accordingly, the atrial (ATR. SENSE) and ventricular (VTR. SENSE) sensing circuits, 482 and 484, may include dedicated sense amplifiers, multiplexed amplifiers or shared amplifiers. The switch 474 determines the “sensing polarity” of the cardiac signal by selectively closing the appropriate switches, as is also known in the art. In this way, the clinician may program the sensing polarity independent of the stimulation polarity. Each sensing circuit, 482 and 484, preferably employs one or more low power, precision amplifiers with programmable gain and/or automatic gain control, automatic sensitivity control bandpass filtering, and a threshold detection circuit, as known in the art, to selectively sense the cardiac signal of interest. The automatic gain/sensitivity control enables device 10 to deal effectively with the difficult problem of sensing the low amplitude signal characteristics of atrial or ventricular fibrillation. The outputs of the atrial and ventricular sensing circuits, 482 and 484, are connected to the microcontroller 460 which, in turn, are able to trigger or inhibit the atrial and ventricular pulse generators, 470 and 472, respectively, in a demand fashion in response to the absence or presence of cardiac activity in the appropriate chambers of the heart.

For arrhythmia detection, device 10 utilizes the atrial and ventricular sensing circuits, 482 and 484, to sense cardiac signals to determine whether a rhythm is physiologic or pathologic. As used in this section “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 460 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 490. The data acquisition system 490 is configured to acquire intracardiac electrogram signals, convert the raw analog data into a digital signal, and store the digital signals for later processing and/or telemetric transmission to an external device 502. The data acquisition system 490 is coupled to the right atrial lead 420, the LV lead 424, and the right ventricular lead 430 through the switch 474 to sample cardiac signals across any pair of desired electrodes. The microcontroller 460 is further coupled to a memory 494 by a suitable data/address bus 496, wherein the programmable operating parameters used by the microcontroller 460 are stored and modified, as required, in order to customize the operation of device 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable device 10 may be non-invasively programmed into the memory 494 through a telemetry circuit 500 in telemetric communication with the external device 502, such as a programmer, transtelephonic transceiver or a diagnostic system analyzer. The telemetry circuit 500 is activated by the microcontroller by a control signal 506. The telemetry circuit 500 advantageously allows intracardiac electrograms and status information relating to the operation of device 10 (as contained in the microcontroller 460 or memory 494) to be sent to the external device 502 through an established communication link 504. Device 10 further includes an accelerometer or other physiologic sensor 508, commonly referred to as a “rate-responsive” sensor because it is typically used to adjust pacing stimulation rate according to the exercise state of the patient. However, the physiological sensor 508 may further be used to detect changes in cardiac output, changes in the physiological condition of the heart, or diurnal changes in activity (e.g., detecting sleep and wake states) and to detect arousal from sleep. Accordingly, the microcontroller 460 responds by adjusting the various pacing parameters (such as rate, AV delay, VV delay, etc.) at which the atrial and ventricular pulse generators, 470 and 472, generate stimulation pulses. While shown as being included within device 10, it is to be understood that the physiologic sensor 508 may also be external to device 10, yet still be implanted within or carried by the patient. A common type of rate responsive sensor is an activity sensor incorporating an accelerometer or a piezoelectric crystal, which is mounted within the housing 440 of device 10. Other types of physiologic sensors are also known, for example, sensors that sense the oxygen content of blood, respiration rate and/or minute ventilation, pH of blood, ventricular gradient, etc. Still further, the sensor may be equipped to detect left atrial pressure (LAP), left ventricular pressure (LVP), right ventricular pressure (RVP), photoplethysmography (PPG) or heart sounds. It should be understood that multiple separate sensors can be provided and, depending upon the parameter to be detected, at least some of the sensor might be positioned external to the device housing.

The device additionally includes a battery 510, which provides operating power to all of the circuits shown in FIG. 8. The battery 510 may vary depending on the capabilities of device 10. If the system only provides low voltage therapy, a lithium iodine or lithium copper fluoride cell typically may be utilized. For device 10, which employs shocking therapy, the battery 510 should be capable of operating at low current drains for long periods, and then be capable of providing high-current pulses (for capacitor charging) when the patient requires a shock pulse. The battery 510 should also have a predictable discharge characteristic so that elective replacement time can be detected. Accordingly, appropriate batteries are employed.

As further shown in FIG. 8, device 10 is shown as having an impedance measuring circuit 512, which is enabled by the microcontroller 460 via a control signal 514. Uses for an impedance measuring circuit include, but are not limited to, detecting cardiogenic impedance for the purposes discussed above; lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; detecting the opening of heart valves; etc. The impedance measuring circuit 512 is advantageously coupled to the switch 574 so that any desired electrode may be used.

In the case where device 10 is intended to operate as an ICD device, it detects the occurrence of an arrhythmia, and automatically applies an appropriate electrical shock therapy to the heart aimed at terminating the detected arrhythmia. To this end, the microcontroller 460 further controls a shocking circuit 516 by way of a control signal 518. The shocking circuit 516 generates shocking pulses of low (up to 0.5 joules), moderate (0.5-10 joules) or high energy (11 to 40 joules or more), as controlled by the microcontroller 460. Such shocking pulses are applied to the heart of the patient through at least two shocking electrodes, and as shown in this embodiment, selected from the left atrial coil electrode 428, the RV coil electrode 436, and/or the SVC coil electrode 14. The housing 440 may act as an active electrode in combination with the RV electrode 436, or as part of a split electrical vector using the SVC coil electrode 14 or the left atrial coil electrode 428 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 4-40 joules), delivered asynchronously (since R-waves may be too disorganized), and pertaining exclusively to the treatment of fibrillation. Accordingly, the microcontroller 460 is capable of controlling the synchronous or asynchronous delivery of the shocking pulses.

The microcontroller includes an on-board Z-based mechanical dyssynchrony assessment system 501 operative to perform or control all or some of the mechanical dyssynchrony assessment techniques described above. Assessment system 501 includes an impedance (Z) vector measurement system 503 operative to measure signals representative of impedance along different RV-LV vectors between an electrode in the RV and the electrodes of the multi-pole LV lead, as well as to measure impedance along vectors between RV electrodes such as RV tip to RV coil. An impedance reference point identification system 505 is operative to identify reference points (Zmax, Zmin) within the measured signals for each of the different RV-LV vectors, as well as for RV vectors such as RV tip to RV coil. A reference point timing determination system 507 is operative to determine relative timings of corresponding reference points within the measured signals of the different RV-LV vectors, as well as between reference points within RV vectors and corresponding points within one or more of the RV-LV vectors. A LV mechanical dyssynchrony determination system 509 is operative to determine values representative of LV mechanical dyssynchrony from a comparison of the relative timings of the corresponding reference points within the measured RV-LV impedance signals. A VV mechanical dyssynchrony determination system 511 is operative to determine values representative of interventricular mechanical dyssynchrony from a comparison of the relative timings of the corresponding reference points within an RV impedance signal and one or more of the RV-LV impedance signals. A stroke volume determination system 513 estimates stroke volume, as discussed above.

The microcontroller also includes an HF detection/warning/CRT/therapy/pacing optimization controller 515 operative to perform or control all or some of the functions described above in response to the detection of mechanical dyssynchrony, such as detecting a tracking HF, generating warnings, controlling CRT, selecting electrodes and optimizing pacing delay parameters (based, for example, on both M_DS and SVZ), etc. An internal warning device 499 may be provided for generating perceptible warning signals to the patient via vibration, voltage or other methods. Diagnostic data may be recorded in memory 494.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

As noted, at least some of the techniques described herein can be performed by (or under the control of) an external device. For the sake of completeness, an exemplary device programmer will now be described, which includes components for controlling at least some of the functions and steps already described.

### Exemplary External Programmer

FIG. 9 illustrates pertinent components of an external programmer 16 for use in programming the device of FIG. 7 and for performing or controlling the above-described mechanical dyssynchrony assessment techniques. For the sake of completeness, other device programming functions are also described herein. Generally, the programmer permits a physician, clinician or other user to program the operation of the implanted device and to retrieve and display information received from the implanted device such as intracardiac electrogram (IEGM) data and device diagnostic data. Additionally, the external programmer can be optionally equipped to receive and display electrocardiogram (ECG) data from separate external surface ECG leads that may be attached to the patient. Depending upon the specific programming of the external programmer, programmer 16 may also be capable of processing and analyzing data received from the implanted device and from the ECG leads to, for example, render preliminary diagnosis as to medical conditions of the patient or to the operations of the implanted device.

Now, considering the components of programmer 16, operations of the programmer are controlled by a CPU 602, which may be a generally programmable microprocessor or microcontroller or may be a dedicated processing device such as an ASIC or the like. Software instructions to be performed by the CPU are accessed via an internal bus 604 from a read only memory (ROM) 606 and random access memory 630. Additional software may be accessed from a hard drive 608, floppy drive 610, and CD ROM drive 612, or other suitable permanent mass storage device. Depending upon the specific implementation, a basic input output system (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 614 or other suitable computer display device. To this end, the CPU may, for example, display a menu of specific programmable parameters of the implanted device to be programmed or may display a menu of types of diagnostic data to be retrieved and displayed. In response thereto, the physician enters various commands via either a touch screen 616 overlaid on the LCD display or through a standard keyboard 618 supplemented by additional custom keys 620, such as an emergency VVI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

Once all pacing leads are mounted and the pacing device is implanted, the various parameters are programmed. Typically, the physician initially controls the programmer 16 to retrieve data stored within any implanted devices and to also retrieve ECG data from ECG leads, if any, coupled to the patient. To this end, CPU 602 transmits appropriate signals to a telemetry subsystem 622, which provides components for directly interfacing with the implanted devices, and the ECG leads. Telemetry subsystem 622 includes its own separate CPU 624 for coordinating the operations of the telemetry subsystem. Main CPU 602 of programmer communicates with telemetry subsystem CPU 624 via internal bus 604. Telemetry subsystem additionally includes a telemetry circuit 626 connected to telemetry wand 628, which, in turn, receives and transmits signals electromagnetically from a telemetry unit of the implanted device. The telemetry wand is placed over the chest of the patient near the implanted device to permit reliable transmission of data between the telemetry wand and the implanted device. Herein, the telemetry subsystem is shown as also including an ECG circuit 634 for receiving surface ECG signals from a surface ECG system 632. In other implementations, the ECG circuit is not regarded as a portion of the telemetry subsystem but is regarded as a separate component.

Typically, at the beginning of the programming session, the external programming device controls the implanted devices via appropriate signals generated by the telemetry wand to output all previously recorded patient and device diagnostic information. Patient diagnostic information includes, for example, recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like. Data retrieved from the device also includes the data stored within the recalibration database of the device (assuming the device is equipped to store that data.) Data retrieved from the implanted devices is stored by external programmer 16 either within a random access memory (RAM) 630, hard drive 608 or within a floppy diskette placed within floppy drive 610. Additionally, or in the alternative, data may be permanently or semi-permanently stored within a compact disk (CD) or other digital media disk, if the overall system is configured with a drive for recording data onto digital media disks, such as a write once read many (WORM) drive.

Once all patient and device diagnostic data previously stored within the implanted devices is transferred to programmer 16, the implanted devices may be further controlled to transmit additional data in real time as it is detected by the implanted devices, such as additional IEGM data, lead impedance data, and the like. Additionally, or in the alternative, telemetry subsystem 622 receives ECG signals from ECG leads 632 via an ECG processing circuit 634. As with data retrieved from the implanted device itself, signals received from the ECG leads are stored within one or more of the storage devices of the external programmer. Typically, ECG leads output analog electrical signals representative of the ECG. Accordingly, ECG circuit 634 includes analog to digital conversion circuitry for converting the signals to digital data appropriate for further processing within the programmer. Depending upon the implementation, the ECG circuit may be configured to convert the analog signals into event record data for ease of processing along with the event record data retrieved from the implanted device. Typically, signals received from the ECG leads are received and processed in real time.

Thus, the programmer receives data both from the implanted devices and from optional external ECG leads. Data retrieved from the implanted devices includes parameters representative of the current programming state of the implanted devices. Under the control of the physician, the external programmer displays the current programmable parameters and permits the physician to reprogram the parameters. To this end, the physician enters appropriate commands via any of the aforementioned input devices and, under control of CPU 602, the programming commands are converted to specific programmable parameters for transmission to the implanted devices via telemetry wand 628 to thereby reprogram the implanted devices. Prior to reprogramming specific parameters, the physician may control the external programmer to display any or all of the data retrieved from the implanted devices or from the ECG leads, including displays of ECGs, IEGMs, and statistical patient information. Any or all of the information displayed by programmer may also be printed using a printer 636.

Additionally, CPU 602 also includes a Z-based mechanical dyssynchrony/stroke volume assessment system 650 operative to perform all or some of the functions of corresponding on-board system 501, discussed above, based on data transmitted to/from the implanted device, particularly the aforementioned mechanical dyssynchrony and stroke volume assessment functions. The microcontroller also includes a programmed-based HF detection/warning/CRT/therapy/pacing optimization controller 652 operative to perform or control all or some of the functions described above in response to the detection of mechanical dyssynchrony and/or stroke volume, such as detecting and tracking HF, generating warnings, controlling CRT, optimizing pacing delay parameters, etc.

Depending upon the implementation, the various components of the CPU may be implemented as separate software modules or the modules may be combined to permit a single module to perform multiple functions. In addition, although shown as being components of the CPU, some or all of these components may be implemented separately using ASICs or the like.

Programmer/monitor 16 also includes an internet connection 638 to permit direct transmission of data to other programmers via the public switched telephone network (PSTN) or other interconnection line, such as a T1 line or fiber optic cable or wireless connection (WiFi). Depending upon the implementation, the internet connection may be connected directly to internal bus 604 may be connected to the internal bus via either a parallel port 640 or a serial port 642. Other peripheral devices may be connected to the external programmer via parallel port 640 or a serial port 642 as well. Although one of each is shown, a plurality of input output (I/O) ports might be provided, including USB ports, etc. A speaker 644 is included for providing audible tones to the user, such as a warning beep in the event improper input is provided by the physician. Telemetry subsystem 622 additionally includes an analog output circuit 645 for controlling the transmission of analog output signals, such as IEGM signals output to an ECG machine or chart recorder.

With the programmer configured as shown, a clinician or other user operating the external programmer is capable of retrieving, processing and displaying a wide range of information received from the implanted device and to reprogram the implanted device if needed. The descriptions provided herein with respect to FIG. 9 are intended merely to provide an overview of the operation of programmer and are not intended to describe in detail every feature of the hardware and software of the programmer and is not intended to provide an exhaustive list of the functions performed by the programmer.

In general, while the invention has been described with reference to particular embodiments, modifications can be made thereto without departing from the scope of the invention. Note also that the term “including” as used herein is intended to be inclusive, i.e. “including but not limited to.”

1. A method for use with an implantable medical device for implant within a patient having a lead system including a right ventricular (RV) lead a multi-pole left ventricular (LV) lead having a plurality of electrodes, the method comprising:
measuring signals representative of impedance (Z) along different RV-LV vectors between an electrode in the RV and the electrodes of the multi-pole LV lead as the signals vary during a cardiac cycle;
identifying reference points within the measured signals for each of the different RV-LV vectors;
determining a reference point within a QRS complex;
determining relative timings from the reference point within the QRS complex to corresponding reference points within the measured signals of the different RV-LV vectors;
determining values, associated with the different RV-LV vectors, representative of mechanical dyssynchrony by comparing the relative timings of the corresponding reference points within the measured signals; and
controlling at least one device function based on the values representative of mechanical dyssynchrony associated with the different RV-LV vectors.

2. The method of claim 1 wherein measuring signals representative of impedance (Z) includes measuring the signals along each of: an LV distal ring electrode-RV electrode vector; an LV mid1 ring electrode-RV electrode vector; an LV mid2 ring electrode-RV electrode vector; and an LV proximal ring electrode-RV electrode vector.

3. The method of claim 2 wherein the RV electrode is one of an RV ring electrode and an RV coil electrode.

4. The method of claim 1 wherein identifying reference points within the measured signals for each of the different RV-LV vectors includes determining a peak of the QRS complex and determining one or more of a maximum impedance (Zmax) point and a minimum impedance (Zmin) point during the cardiac cycle for each of the different vectors.

5. The method of claim 1 wherein identifying reference points within the measured signals for each of the different RV-LV vectors includes determining a maximum rate of change in impedance (max dZ/dt) point during the cardiac cycle for each of the different RV-LV vectors and wherein the values representative of mechanical dyssynchrony are determined from a comparison of the relative timings of the max dZ/dt points within the measured signals.

6. The method of claim 1 wherein determining values representative of mechanical dyssynchrony additionally includes determining values representative of a cross-correlation between the measured signals of each of the different RV-LV vectors.

7. The method of claim 1 wherein controlling at least one device function based on the values representative of mechanical dyssynchrony includes controlling pacing therapy to address heart failure.

8. The method of claim 1 wherein controlling at least one device function based on the values representative of mechanical dyssynchrony includes controlling LV interelectrode pacing delay values.

9. The method of claim 1 including detecting stroke volume from the signals representative of impedance (Z) and wherein the method further includes controlling one or more of atrioventricular (AV) pacing delay values and interventricular (VV) pacing delay values based on one or more of mechanical dyssynchrony and stroke volume.

10. The method of claim 9 wherein one or more of AV pacing delay values and VV pacing delay values are adjusted to achieve one or more of minimizing mechanical dyssynchrony and maximizing stroke volume.

11. The method of claim 9 wherein one or more of AV pacing delay values and VV pacing delay values are adjusted to achieve an acceptable range of both mechanical dyssynchrony and stroke volume.

12. The method of claim 1 wherein one or more of AV pacing delay values and VV pacing delay values are adjusted based on intracardiac electrogram (IEGM) signals in combination with the values representative of mechanical dyssynchrony.

13. The method of claim 1 wherein one or more of electrodes are selected for delivering pacing therapy based on the values representative of mechanical dyssynchrony.

14. The method of claim 1 wherein one or more of electrodes are selected for delivering pacing therapy based on the values representative of mechanical dyssynchrony in combination with intracardiac electrogram (IEGM) signals.

15. The method of claim 1 wherein at least some of the steps are performed by an external device based on signals received from the implantable medical device.

16. The method of claim 1 wherein the measured impedance signals include impedance signals sensed along RV-RV vectors with current injected from an RV electrode to an LV electrode.

17. The method of claim 16 wherein the RV-RV sensing vectors include one or more of RV tip to RV coil and RV ring to RV coil.

18. The method of claim 16 wherein the current injection vector includes RV coil to LV tip.

19. A system for use with an implantable medical device for implant within a patient having a lead system including a right ventricular (RV) lead a multi-pole left ventricular (LV) lead having a plurality of electrodes, the system comprising:
an RV-LV impedance (Z) vector measurement system operative to measure signals representative of impedance along different RV-LV vectors between an electrode in the RV and the electrodes of the multi-pole LV lead as the signals vary during a cardiac cycle;
an impedance reference point identification system operative to identify reference points within the measured signals for each of the different RV-LV vectors;
a reference point timing determination system operative to determine a reference point within a QRS complex and relative timings from the reference point within the QRS complex to corresponding reference points within the measured signals of the different RV-LV vectors; and
a mechanical dyssynchrony determination system operative to determine values, associated with the different RV-LV vectors, representative of mechanical dyssynchrony by comparing the relative timings of the corresponding reference points within the measured signals associated with the different RV-LV vectors.

20. A system for use with an implantable medical device for implant within a patient having a lead system including a right ventricular (RV) lead a multi-pole left ventricular (LV) lead having a plurality of electrodes, the system comprising:
means for measuring signals representative of impedance (Z) along different RV-LV vectors between an electrode in the RV and the electrodes of the multi-pole LV lead as the signals vary during a cardiac cycle;
means for identifying reference points within the measured signals for each of the different RV-LV vectors;
means for determining a reference point within a QRS complex and determining relative timings from the reference point within the QRS complex to corresponding reference points within the measured signals of the different RV-LV vectors; and
means for determining values, associated with the different RV-LV vectors, representative of mechanical dyssynchrony by comparing the relative timings of the corresponding reference points within the measured signals associated with the different RV-LV vectors.
